(19) United States
(12) Patent Application Publication
(10) Pub. No.: US 2025/0065149 A1
(43) Pub. Date: Feb. 27, 2025

(54) ADMINISTRATION SYSTEM

(71) Applicant: QUIREM Medical B.V., Deventer (NL)

(72) Inventor: Gradus VAN WOLFSWINKEL, Barneveld (NL)

(73) Assignee: QUIREM Medical B.V., Deventer (NL)

(21) Appl. No.: 18/729,485

(22) PCT Filed: Jan. 17, 2023
(86) PCT No.: PCT/NL2023/050021

§ 371 Date: Jul. 16, 2024

(30) Foreign Application Priority Data
Jan. 17, 2022 (NL) 2030571

(51) Int. Cl. A61N 5/10; A61M 5/178; A61M 5/20

(52) U.S. Cl. CPC A61N 5/1007; A61M 5/2053; A61M 2005/1787; A61N 2005/1021

(57) ABSTRACT

A system (1) for administration of a mixture of particles, the system comprising an administration subsystem (3) comprising at least one administration hydraulic element (14) for containing a mixture with the particles, wherein the at least one administration hydraulic element is movably mounted for, when moving, homogenizing the mixture, wherein said at least one administration hydraulic element is arranged for administering of the mixture to a further system (15), an actuation subsystem (2) comprising at least one actuation hydraulic element (4); wherein the at least one actuation hydraulic element is operably connected to the at least one administration hydraulic element for actuating said administration hydraulic element to administer mixture to the further system; wherein the at least one actuation hydraulic element is further fluidly connected with an operating element (5) to operate the at least one actuation hydraulic element.

This application is a National Stage application under 35 U.S.C. 371 of International Patent Application Serial No. PCT/NL2023/050021, filed Jan. 17, 2023, which claims the benefit and priority of Netherlands Patent Application No. 2030571, filed Jan. 17, 2022, the disclosures of all are incorporated herein by reference in their entirety.

The invention relates to an administration system.

Such administration system can be used for administering radio-active particles to a patient. These administration systems are known and can be used in hospitals for the treatment of patients, e.g. of patients with tumors.

Radio-active particles are typically contained in a vial. The radio-active particles are also referred to as microspheres, or to activity in general. The radio-active particles need to be administered to a patient via a suspension fluid in order to enable transferal of the particles along an administration line. Administration systems are known in which the vial is coupled to a bag with flushing fluid, the flushing fluid is used to bring the microspheres in the vial in a suspension allowing the suspension to be administered. By flushing the vial with the flushing fluid, the microspheres in the vial are brought in suspension and then directly administered to the patient. The vial is coupled to an administration line via which the suspension is administered into a catheter into the patient. The vial contains in that case the total planned amount of activity needed for that specific treatment. Flushing fluid can be added to the vial by using an operating element. For example, the operating element can e.g. be a valve that can be opened or closed by an operator, wherein the operating element can be connected between the vial and the flushing fluid bag. However, a drawback of such systems is that administration of precise doses of radioactivity is not possible. Only after a vial has been completely emptied, the administered dose is exactly known. Consequently, all the microspheres in the vial are administered to the same location at the patient. It is difficult to control the precise amount, or volume, of radio-active particles that is administered. Only when the entire vial is empty, the operator knows the amount of activity administered to the patient. It is not possible to serve multiple locations at the patient with a single vial. Then, multiple vials are required. Additionally, in known administration systems, operation of the operating element e.g. the valve by the operator is done in proximity of the radio-active suspension.

Also, administration systems are known in which a vial is prepared containing the radio-active particles as well as the suspension fluid to provide a suspension then containing the total amount of radio-active particles and suspension fluid to obtain the required concentration of radio-active particles. In some administration systems, such a vial is being rotated to provide an approximately homogenous suspension. When a homogenous suspension is obtained, the suspension is administered to the patient. Various operating elements can be used to that end, often a rotatable knob is used that moves a spindle connected to the vial to gradually empty the vial. However, such operating elements require proximity of the operator to the radio-active suspension. Also, administering the required amount of suspension from a rotating vial remains cumbersome, inaccurate, inconvenient and unpredictable.

There is a need for an administration system that allows more accurate administration of particles, also referred to as activity or microspheres, to a further system such as a patient. Also, there is a need for an administration system allowing an operator to more safely operate the administration in an intuitive manner. As modern medical treatments can comprise administration in conjunction with real-time magnetic resonance imaging (MRI), there is also an additional need for MRI compatible administration systems.

It is an object of the invention to provide for an administration system that alleviates at least one of the above mentioned drawbacks.

Thereto, the invention provides for a system for administration of particles, the system comprising an administration subsystem and an actuation subsystem. The administration subsystem comprises at least one administration hydraulic element for containing a mixture with the particles, wherein the at least one administration hydraulic element is movably mounted for, when moving, homogenizing the mixture with the particles, wherein said at least one administration hydraulic element is arranged for administering of the mixture to a further system. The actuation subsystem comprises at least one actuation hydraulic element, wherein the at least one actuation hydraulic element is operably connected to the at least one administration hydraulic element for actuating said administration hydraulic element to administer mixture to the further system, wherein the at least one actuation hydraulic element is further fluidly connected with an operating element to operate the at least one actuation hydraulic element.

By providing two separate subsystems, a subsystem for actuation and a subsystem for administration, a safer use of the administration system may be possible. Also, it may provide for a more modular administration system, for example when the actuation subsystem can be coupled to various administration subsystems. As such, one may prepare an administration subsystem, e.g. depending on the further system to be connected to, and/or depending on the use of the mixture to be administered. When the actual administration then is to be prepared, the actuation subsystem may be coupled to one of the administration subsystems.

The system comprises hydraulic elements, e.g. syringes, or any other hydraulic elements, e.g. including a piston in a barrel. Instead of hydraulic elements, the administration system can also be envisaged to comprise pneumatic elements. By providing hydraulic elements, in particular syringes, use can be made of components that are known and approved for clinical use. Also, by providing hydraulic elements, operation thereof can be relatively simple. Advantageously, the hydraulic elements of the actuation subsystem may comprise water. Since the actuation subsystem is, hydraulically, decoupled from the administration subsystem, a different type of fluid can be used in the administration subsystem and in the actuation subsystem. In another example, gas or pressurized gas may be used to fill the hydraulic elements of the actuation subsystem.

The at least one administration hydraulic element is arranged for receiving and containing the mixture with the particles. The particles can e.g. be radio-active particles, microspheres such as QuiremSpheres, SIR-Speres, TheraSphere or CT imageable microspheres, or microspheres labeled with chemotherapeutic agents, such as TACE microspheres, or the particles can be nanoparticles of 20-1000 nanometer or 1-1000 micrometer, more preferably 1-400 micrometer, more preferably 10-60 or 60-200 micron, or 80-150 micron for embolization of vessels or any other type of particle that is to be administered via the mixture. The mixture with the particles can be a fluid/fluid mixture, or a fluid/solid mixture or a solid/solid mixture. Advantageously, the mixture is a fluid/solid suspension. It may be envisaged that, depending on the particles, a mixture of gas/solid or gas/fluid is provided.

The at least one administration hydraulic element is movably mounted, i.e. mounted such that it allows movement, e.g. a rotational, swiveling, vibrating or translation movement, or a combination thereof, or any other movement that can increase homogeneity of the mixture held within the at least one administration hydraulic element. By mounting the hydraulic element in a movable arrangement, the mixture in the hydraulic element can be brought in motion as well. By bringing the mixture in the hydraulic element in motion, the mixture can become homogeneous. When a homogenous distribution of the particles in the mixture is obtained, the amount of particles in a predefined volume of mixture can be determined. As such, obtaining a homogeneous mixture, may allow for an accurately controlled and more predictable administration of a predetermined amount of particles.

The administration subsystem provides for administration of the mixture to a further system, and provides compatibility for different uses. The further system can be a clinical system, e.g. an intravenous system, an intra-tumoral system, or an intra-arterial system, or an industrial system, e.g. for filling multiple vials simultaneously with the mixture containing particles, or any other system configured to receive the mixture from the administration system. As such, the administration system can be used in a versatile manner.

By actuating the administration hydraulic element, mixture that is contained in the administration hydraulic element is discharged from the administration hydraulic element, to the further system.

By providing an actuation subsystem that is operably connected to the at least one administration hydraulic element, actuation of the administration hydraulic element can be done in a safe, sometimes even remote manner. The actuation subsystem comprises at least one actuation hydraulic element, which actuation hydraulic element is operably connected to the administration hydraulic element. By actuating the actuation hydraulic element, the administration hydraulic element is actuated as well, and the mixture contained in the administration hydraulic element is outputted out of the administration hydraulic element, preferably to the further system that is connected to the administration subsystem. By providing an actuation hydraulic element, the actuation can be done relatively accurate and/or precise. By operably connecting the actuation hydraulic element with the administration hydraulic element, the actuation of the actuation hydraulic element can be transferred, preferably directly, to the administration hydraulic element. As such any losses due to the operable connection between the actuation hydraulic element and the administration hydraulic element can be minimized or obviated.

The administration and actuation hydraulic elements are advantageously operably connected, e.g. mechanically connected, such as (partially) directly pressed together or connected by interconnection means, electronically connected or connected in another way. By operably connecting the at least one actuation hydraulic element with the at least one administration hydraulic element, it can be obtained that actuation of the one results in actuation of the other. The connection can e.g. be a mechanical connection, such that e.g. actuation of a plunger of the actuation hydraulic element directly moves a plunger of the administration hydraulic element, or the connection can be electronically, such that e.g. actuation of an actuation hydraulic element is transformed into an electronic or an electric signal that, wired or wireless, is transmitted to the administration hydraulic element and is converted into a mechanical actuation of the administration hydraulic element for example by means of an actuator.

By further providing an operating element, that operates the at least one actuation hydraulic element, the actuation hydraulic element can be actuated in a relative accurate manner. For example, the actuation hydraulic element can be a syringe, and the operating element can be another syringe fluidly connected with the actuation syringe, such that by actuating the operating syringe, the actuation syringe is operated as well. For example, the operating element can be of a much smaller volume than the actuation hydraulic element allowing an accurate operation. Alternatively, the operating element can be electronically or electrically connected to the administration hydraulic element. For example, actuation of the operating element is transformed into an electronic or an electric signal that, wired or wireless, is transmitted to the administration hydraulic element and moves the plunger of the administration hydraulic element. As such, the operating element can be remote from the administration subsystem and/or from the actuation hydraulic element, which can provide for a safe use of the system in particular in an MR (magnetic resonance)-environment. An MR-environment can be for example an environment in which an MRI-system is positioned. The operating element can then e.g. be brought outside of the MR-environment. In an embodiment, it can be envisaged that the operating element electronically and/or electrically operates the administration hydraulic element via an electronic and/or electrical signal, thereby obviating the actuation hydraulic element.

Alternatively, the operating element may have the same or a smaller volume than the actuation hydraulic element. When fluidly coupling the operating element with the actuation hydraulic element, it can be advantageous to provide a more precise operating element having a smaller volume than the actuation hydraulic element. In particular when the operating element is a syringe it can be advantageous to provide a small volume syringe, e.g. 1 ml, to actuate a larger volume actuation hydraulic element e.g. a 20 ml syringe. This allows for a more accurate actuation.

In order to advantageously provide feedback to an administrator of the system, the actuation subsystem can further comprise a dosage hydraulic element, e.g. a read-out syringe, fluidly connected to the at least one actuation hydraulic element. By fluidly connecting the dosage hydraulic element with the at least one actuation hydraulic element, the dosage hydraulic element can be filled/emptied at the same flow rate of emptying the at least one actuation hydraulic element. Feedback information is provided to an administrator by means of a scaling fixed onto or placed next to the dosage hydraulic element. In case a scaling is not directly attached to the dosage hydraulic element, the dosage hydraulic element and/or the scaling can be fixed to a surface in order to eliminate variations due to movements. Alternatively, the matter volume present inside the dosage hydraulic element can be electronically sensed and can be shown on a display which is monitored by the administrator. Other means of conveying information to the administrator are also possible.

The actuation subsystem can further comprise an actuation valve unit, the actuation valve unit fluidly connecting the operating element and the at least one actuation hydraulic element and/or the dosage hydraulic element. The actuation valve unit enables transportation of a fluid the operating element and the at least one actuation hydraulic element. By enabling transportation of a fluid from the operating element to the at least one actuation hydraulic element, the administration subsystem can be actuated with the operating element. The actuation valve unit can also enable transportation of a fluid between the at least one actuation hydraulic element and the dosage hydraulic element in order to provide the abovementioned fluid connection between said elements. The actuation valve unit can also enable transportation of a fluid between the dosage hydraulic element and the operating element. The actuation valve unit can advantageously provide means to set which of the abovementioned direction(s) are active to determine how a fluid will flow when any of the hydraulic elements are actuated in order to easily operate the system during different stages of use, such as set-up and administration.

Further, the actuation valve unit can comprise at least one three way valve. The three way valve allows for a compact hydraulic connection between the operating element, the at least one actuation hydraulic element and/or the dosage hydraulic element. The three way valve allows fluid flow to alternate in at least two directions through a same connection, resulting in an efficient transportation of a fluid and a minimization of necessary flow lines. Additionally, the at least one three way valve may contribute to ease of use of the administration system by providing user-friendly means, e.g. rotary knob, sliders, buttons or the like, to easily switch between flow directions by manual operation. Advantageously, automated operation can be implemented to further contribute to the ease of use of the administration system, by providing an interface through which the three way valve can be set by an administrator. This way, an administrator can be located at a distance from the administration system while still having full control over the administration system. Alternatively, fully automated operation of the three way valve can be provided, wherein no human interaction is required to set up the three way valve in the different stages of use of the administration system.

The actuation valve unit can advantageously comprise a bypass line between the at least one actuation hydraulic element and the dosage hydraulic element to allow a direct fluid connection between the at least one actuation hydraulic element and the dosage hydraulic element. The direct fluid connection between the at least one actuation hydraulic element and the dosage hydraulic element can enable a simultaneous and equal fluid flow to both said hydraulic elements when the operating element is actuated in use, in order to concurrently fill the dosage hydraulic element as the at least one actuation hydraulic element is being emptied. Thereto, the bypass line can have a length such that a distance from the operating element to the dosage hydraulic element is the same as a distance from the operating element to the actuation hydraulic element. This may simplify the integration and the operation of the dosage hydraulic element in the actuation subsystem.

The operating element can be a hydraulic element, increasing precision of administration and contributing to the uniformity of the system. In an embodiment, the operating element is a syringe. A syringe as operating element significantly improves the actuation accuracy. In addition, as an administrator of the system can be expected to be a medically educated person, therefore professionally trained in syringe handling skills, the use of a syringe as the operating element increases the ease of use of the system and reduces time necessary for learning how to operate the system and/or may reduce errors in handling of the operating element. Additionally, the use of a syringe as the operating element makes the experience for an administrator similar to an administration wherein a patient is located right next to the administrator. This effect makes the experience more familiar for and improves the control of an administrator.

By providing the operating element such that it contains a narrower cylinder-like volume than the actuation hydraulic element, the precision of administration can be greatly increased, e.g. by providing an operating element as a syringe with a diameter smaller than a diameter of the at least one actuation syringe. A piston of the operating element will then move a smaller volume of fluid for a same translation along the syringe container, thus increasing accuracy. A scaling fixedly attached on the operating element will provide a better accuracy than if the diameter of the operating element were the same or larger than the diameter of the actuation hydraulic element. Alternatively, a scaling means can be placed next to the operating element and the operating element and/or the scaling can be fixed to a surface in order to eliminate variations due to movements.

Advantageously, the operating element can have a volume smaller than a volume of the at least one actuation hydraulic element, preferably can have a volume that is about 10 times smaller than the volume of the actuation hydraulic element, more preferably can have a volume that is about 20 times smaller than the volume of the actuation hydraulic element, enabling the operating element to, in use, completely empty the volume of a filled actuation hydraulic element in multiple steps, preferably 10 steps, more preferably 20 steps, wherein one step comprises completely emptying a filled operating element. The precision of administration can be greatly increased by providing an operating element, e.g. as a syringe, with a diameter smaller than a diameter of the at least one actuation syringe. The smaller the diameter, the more displacement of e.g. a plunger of a syringe is needed to obtain the same volume, which may result in a more precise control of the volume to be administered. A scaling fixedly attached thereon, may then provide a better accuracy than if the diameter of the operating element is the same or larger than the diameter of the actuation hydraulic element. Alternatively, a scaling means can be placed next to the operating element and the operating element and/or the scaling can be fixed to a surface in order to eliminate variations due to movements.

Alternatively, the matter volume present inside the operating element can be electronically sensed and can be shown on a display which is monitored by the administrator. Other means of conveying information to the administrator are also possible.

The dosage hydraulic element can further have a volume that is the same or larger than a volume of the at least one actuation hydraulic element, enabling the dosage hydraulic element to indicate the volume of fluid left in the at least one actuation hydraulic element. These volume relations can be provided by the actuation subsystem in order to enable concurrently filling or emptying the dosage hydraulic element during completely emptying a fully filled actuation hydraulic element into the administration subsystem. This way, information concerning the remaining volume inside the actuation hydraulic element can be provided to an administrator during the entire period of time of emptying a fully filled actuation hydraulic element, without a need for intermediately resetting the dosage hydraulic element. Advantageously, the dosage hydraulic element and the actuation hydraulic element can have the same volume in order to prevent the dosage hydraulic element from having an unused volume part and in order to minimize a size of the dosage hydraulic element. The dosage hydraulic element and the actuation hydraulic element are preferably fluidly connected, and fluid inputted to the actuation hydraulic element for actuation is taken from the dosage hydraulic element and vice versa. By providing the dosage hydraulic element with the same or larger volume than the actuation hydraulic element, it can be ensured that the actuation hydraulic element can be entirely emptied, or filled, during actuation. As such, the administration hydraulic element, actuatable connected to the actuation hydraulic element, can be completely emptied. Advantageously, the actuation hydraulic element and the administration hydraulic element connected thereto, have the same volume. For example, when syringes are used as hydraulic elements, the actuation hydraulic element and the administration hydraulic element are the syringes of the same volume. As such, an accurate outputting of a predetermined volume out of the administration hydraulic element can be obtained.

The operating element and/or the dosage hydraulic element, e.g. a syringe, can be manually operable for proper controllability by a trained administrator. Alternatively, the operating element and/or the dosage hydraulic element can be a syringe pump for an even more accurate administration that can be set beforehand to constant rates of administration during pre-set intervals.

The actuation subsystem can comprise flexible flow lines, e.g. medical tubes, for connection between at least the at least one actuation hydraulic element, the operating element and/or the dosage hydraulic element. The flexible flow lines enable transportation of a fluid between at least the at least one actuation hydraulic element, the operating element and/or the dosage hydraulic element. If medical tubes are used, required restrictions to materials used in medical environments are complied with.

Further, the operating element can be fluidly connected to the at least one actuation hydraulic element via a flexible flow line, wherein the flexible flow line is at least 0.2 m, preferably more than 0.5 m. By creating a distance between the at least one actuation hydraulic element and the operating element, an administrator is located further away from radio-active radiation radiated from the administration subsystem, increasing safety. In addition, with respect to user-friendliness, it is advantageous not to directly connect the different hydraulic elements and the actuation valve unit to each other, but to leave some space in between.

The at least one actuation hydraulic element and/or the dosage element can be syringes in order to contribute to a uniform administration system. By providing the actuation hydraulic element as a syringe, actuation transfer to the administration subsystem is converted from a fluid flow to a piston translation movement, an input with which any administration subsystem could be easily made compatible as known to the skilled person. By providing the dosage element as a syringe, administration information is provided to an administrator in a convenient and well-known format. Advantageously, the actuation hydraulic element and the dosage elements can be identical syringes. Also, syringes are known in medical environments and are approved for medical use, allowing easy implementation of the system in medical and/or clinical environments. Using a syringe as dosage element greatly improves the intuitive use of the operation of the system.

The actuation subsystem can advantageously be filled with a fluid, in particular a liquid, and/or is re-usable. As liquids, for example water, are known to have a very low compressibility, administration of a volume at the operating element can be assumed to correspond to that same volume at the administration subsystem. Another medium with negligible compressibility around room temperature can also be used instead of water. Reusability of the actuation subsystem reduces preparation time before every new administration, as well as material costs.

The administration subsystem can comprise a receiving unit for receiving the at least one administration hydraulic element, wherein the receiving unit is arranged for allowing movement of the at least one administration hydraulic element. The allowed movement can e.g. be a rotational, swiveling or translation movement. The movement enables homogenizing the content of the at least one administration hydraulic element. When the content is made homogenous, the distribution of the particles in the mixture is even over the volume of the mixture. As such, the distribution of the particles in the mixture can be known and the number of particles per unit of volume can be known. Thus, when outputting a certain volume of homogenous mixture, it can be known how much particles are outputted as well. Thus allowing for an accurate administering of particles to the further system.

The receiving unit can further comprise a receiving container for receiving the administration hydraulic element therein, preferably, wherein a wall of the container is provided as a shield against radio-active radiation to ensure safety for persons in the vicinity of the system. The container can provide stability to the administration hydraulic element.

In a receiving opening of the receiving container a drive element can be provided that is arranged for engaging with the administration hydraulic element in order to fasten and shield the administration hydraulic element to further stabilize the administration hydraulic element and to further protect against radio-active radiation.

Advantageously, the at least one administration hydraulic element can be rotatably arranged, e.g. via a rotatable connector, which enables the administration hydraulic element to move so as to homogenize its content by a regular, rotational movement. The movement can be provided by one or more bearings, or other holding elements that allow rotational movement, which hold the administration hydraulic element in place while allowing rotational movement. Rotational movement induces a centrifugal effect, wherein particles are pushed towards the inner surface of the administration hydraulic element, resulting in a more homogenized mixture.

The administration subsystem can comprise a drive unit for driving the movement of the at least one administration hydraulic element to provide for a constant and automated movement. By providing a drive unit, the movement can be regulated accurately and the mixture contained in the at least one administration hydraulic element can be kept approximately homogenous. A movement speed can be set to be constant or can be set to be different during subsequent intervals. A control unit can be installed to monitor the real-time movement speed during operation of the administration system. The drive unit can comprise e.g. a DC motor, an AC motor, a motor including conversion means to convert the rotational movement provided by the motor into a translation movement, or any other suitable drive mechanism compatible with the administration subsystem.

The drive unit can further be drivingly coupled to the drive element to drive the drive element for a simple and compact implementation of the movement, since the drive element is easily reachable from outside the container.

The administration subsystem can advantageously comprise a support frame, preferably comprising the receiving unit and the drive unit, in order to provide stability at the interface between the actuation subsystem and the administration subsystem to enable complete transmission of actuation, which adds to the accuracy of the administration system.

The administration subsystem can further comprise at least one holder for holding a vial initially filled with particles, wherein the vial is fluidly couplable with the administration hydraulic element for loading the particles to the administration hydraulic element, preferably wherein the at least one holder is arranged on the support frame to keep it fixed. By providing a holder for holding a vial, a wall of the holder can be arranged as a shield around the vial to limit radio-active radiation outside the holder. The holder also stabilizes the vial, which is important considering safety seeing that it may contain radio-active particles. Because the vial is fluidly couplable with the administration hydraulic element, e.g. by a flexible flow line, the content of the vial can be directly loaded into the system, without having to first empty the vial content into the administration hydraulic element in a set-up step before use.

Further, the administration subsystem can comprise an administration valve unit for providing a fluid connection between the at least one administration hydraulic element, the vial and the further system. The administration valve unit enables transportation of a fluid between the at least one administration hydraulic element, the vial and the further system, and regulates flow direction by providing user-friendly means, e.g. rotary knob, sliders, buttons or the like, to easily switch between flow directions.

The administration subsystem can further comprise a flushing system that is fluidly couplable to the at least one administration hydraulic element, preferably wherein the flushing system is connectable to the administration valve unit. The flushing system enables removal of radio-active particles from the administration system and possibly from the further system. Flushing can be enabled through e.g. a rotary know, a slider, a button or the like, included by the administration valve unit.

The flushing system can further comprise a flushing hydraulic element and a container containing fluid, e.g. a saline, a contrast agent, a fluid containing drug, a phosphate buffered saline (PBS), etc., wherein the container and the flushing hydraulic element are fluidly connectable to each other, preferably wherein the flushing hydraulic element is connectable to the administration valve unit. Advantageously, the container can be the same container as coupled to the vial for preparing the mixture in the administration syringe, which simplifies the configuration and use of the system.

The administration valve unit comprises at least one three way valve. The three way valve allows flow to alternate in two directions through a same connection, resulting in an efficient transportation of a fluid. Additionally, the at least one three way valve contributes to ease of use of the administration system by providing means to easily switch between flow direction.

The system can further comprise an operable connection between the at least one actuation hydraulic element and the at least one administration hydraulic element, wherein the operable connection comprises a coupling element. Operably connecting the administration hydraulic element and the actuation hydraulic element enables transmission of actuation from the actuation subsystem to the administration subsystem. By providing a coupling element, the administration hydraulic element and the actuation hydraulic element are kept together, which is a simple way to enable actuation transferal between said hydraulic elements.

The coupling element can comprise a first engagement surface for engaging with the actuation hydraulic element, preferably with a piston of the actuation hydraulic element, and a second engagement surface for engaging with the administration hydraulic element, preferably with a piston of the administration hydraulic element, wherein the second engagement surface movable arranged with respect to the first engagement surface. The coupling element fastens the actuation hydraulic element and the administration hydraulic element, forming the interface and connection between the actuation subsystem and the administration subsystem. By engaging with the hydraulic elements with the respective pistons, a translation movement of either piston can cause a translation movement of the other piston.

The coupling element can further be a disc shaped element with the first engagement surface at one side and the second engagement surface at the other side of the disc shaped element, preferably wherein the second engagement surface is rotatably arranged with respect to the first engagement surface to allow rotatable movement of the associated administration hydraulic element. By providing the coupling element as a disc shaped element, the interface between actuation subsystem and administration subsystem is reduced to a simple, easy to install, compact element. By allowing rotatable movement of the administration hydraulic element, the coupling element fulfills the desired requirement of conveying actuation of the actuation hydraulic element to the administration hydraulic element while allowing the administration hydraulic element to rotate or move differently.

The coupling element can be arranged to provide shielding of the administration subsystem, preferably of radio-active radiation of the administration subsystem to further ensure safety for persons in the vicinity of the system.

Further, the coupling element can be configured as a disc-shaped element comprising a ceramic bearing for allowing rotational movement of the second engagement surface as ceramics have excellent wear-resistant properties. Other materials, such as composites, could also be used.

The support frame can further comprise a support extension, wherein the support extension is arranged to support the at least one actuation hydraulic element for engagement with the at least one administration hydraulic element. By providing a support extension, the actuation hydraulic element is stabilized to further contribute to a maximal actuation transferal.

Advantageously, the administration subsystem can be MRI-compatible, in particular a drive unit of the administration subsystem can be MRI-compatible to enable use of the administration system during MRI imaging e.g. in a hospital. The administration of radio-active particles can then be monitored closely through the simultaneous image formation.

The administration subsystem can comprise disposable components. For safety reasons, any components that come into contact with the radio-active particles are provided such that they can easily be removed and disposed. In addition, components that come into contact with the further system might need to be disposable anyways, even if the administration mixture does not comprise radio-active particles.

The administration subsystem can comprise a battery pack for powering the drive unit. By providing a battery pack, using an adapter when drawing power from a mains supply and thereby possibly introducing relevant noise power is being avoided. Using a battery pack is especially advantageous when the administration system is used in the context of an MRI compatible application, seeing that MRI is sensitive to these kinds of external radiofrequencies (RF).

The invention further provides a method for administering a mixture containing particles to a further system. The method comprises providing the system according to the invention, preparing the at least one administration hydraulic element with the mixture containing the particles moving the said administration hydraulic element until the mixture is homogeneous, operating the actuation subsystem to actuate the administration hydraulic element such that a predefined volume of mixture is administered to the further system. Optionally, the method further comprises flushing the mixture through the further system with flushing fluid, e.g. for medical administration to a patient.

The invention further provides a mixture containing particles for use in a method of treating a tumor in an individual, wherein said mixture is administered by a system according to the invention.

The invention further provides a use of a mixture containing particles for the treatment of a tumor in an individual, wherein said mixture is administered by a system according to the invention.

The invention further provides a method of treating an individual with a mixture containing particles, said method comprising providing a system according to the invention, preparing the at least one administration hydraulic element with the mixture containing the particles, moving the said administration hydraulic element until the mixture is homogeneous, operating the actuation subsystem to actuate the administration hydraulic element such that a predefined volume of mixture is administered to the individual, and flushing the mixture through the further system with flushing fluid, thus administrating the mixture to the individual.

The term “tumor” or “cancer”, as used herein, refers to a disease or disorder resulting from the proliferation of oncogenically transformed cells. Said tumor preferably is a liver tumor, pancreas tumor or head and neck tumor. As used herein, the term “liver tumor” refers to a tumor arising in the liver, of any histological type, including but not limited to hepatocellular carcinoma, fibrolamellar carcinoma and cholangiocarcinoma. As used herein, the term “pancreas tumor” refers to a cancer arising in the pancreas of any histological type, including but not limited to exocrine tumors and endocrine tumors. A pancreas tumor may be an adenocarcinoma, acinar cell carcinoma, intraductal papillary-mucinous neoplasm, mucinous cystadenocarcinoma, glucagonoma, insulinoma or multiple endocrine neoplasia type-1. As used herein, the term “head and neck tumor” refers to a cancer arising in the tissues and organs of the head and neck. This term includes cancers of the larynx, throat, lips, mouth, nose and salivary glands, of any histological types. A head and neck tumor may be a squamous cell cancer (squamous cell carcinoma), adenocarcinoma or sarcoma.

The mixture containing particles for administration to an individual can be administered intravenously, intra-tumoral or intra-arterial, depending on the further system being an intravenous system, an intra-tumoral system, or an intra-arterial system.

The particles for administration to an individual for treatment of a tumor can be radio-active, preferably selected from particles comprising Yttrium 90 (e.g. TheraSphere® or SIR-Speres® Y-90) or Holmium 166 (e.g. QuiremSpheres® microspheres). Said radio-active particles are well known for their use in the treatment of chemo-resistant and unresectable tumors such as e.g. liver tumors. Yttrium 90 and Holmium 166 are beta-emitters, that after administration accumulate in the tumor, resulting in a local radiation dose to the tumor whilst sparing healthy liver tissue. The particles for administration to an individual can be CT imageable microspheres. These imageable microspheres may allow to evaluate the spatial distribution in the target tissue. The particles for administration to an individual for treatment of a tumor can be further comprising a chemotherapeutic agent, preferably said chemotherapeutic agent is coupled to the particles such as in transarterial chemoembolization (TACE). TACE uses drug eluting beads loaded with chemotherapeutic agents, such as e.g. Doxorubicin and Irinotecan, that are progressively released into the tumor. The main advantages of TACE over conventional chemotherapy are less systemic toxicity and a better patient tolerance.

The dosage of the particles that are administered to an individual in order to treat a tumor is usually patient specific and depends on factors such as the volume of the targeted tumor, the kind of tumor etc. In radiotherapy dose calculation is often performed before administration. For example, one treatment with QuiremSpheres® particles on average comprises 10 million to 30 million particles, one treatment with SIR-Speres® particles on average comprises 30 million to 60 million particles and one treatment with TheraSphere® particles on average comprises 1.2 million to 8 million particles. In the case of administration of particles comprising chemotherapeutic agents, the dose will depend on which chemotherapeutic agent is administered and which tumor is targeted. As an example, for a small liver tumor, a treatment strategy can comprise doxorubicin doses up to 75 mg per single TACE, while for more advanced or larger tumors the doxorubicin dose can be escalated to up to 150 mg per single TACE.

These and other aspects will further be elucidated with reference to a drawing. The drawing comprises figures of exemplary embodiments. In the drawing:

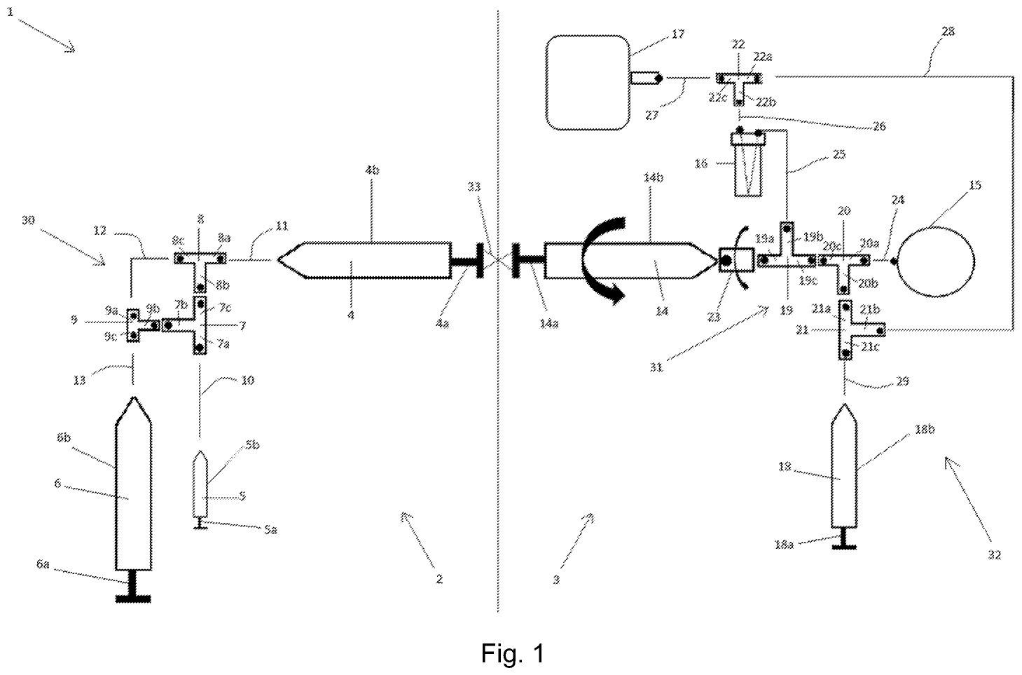

FIG. 1 shows a schematic diagram of an administration system according to the invention;

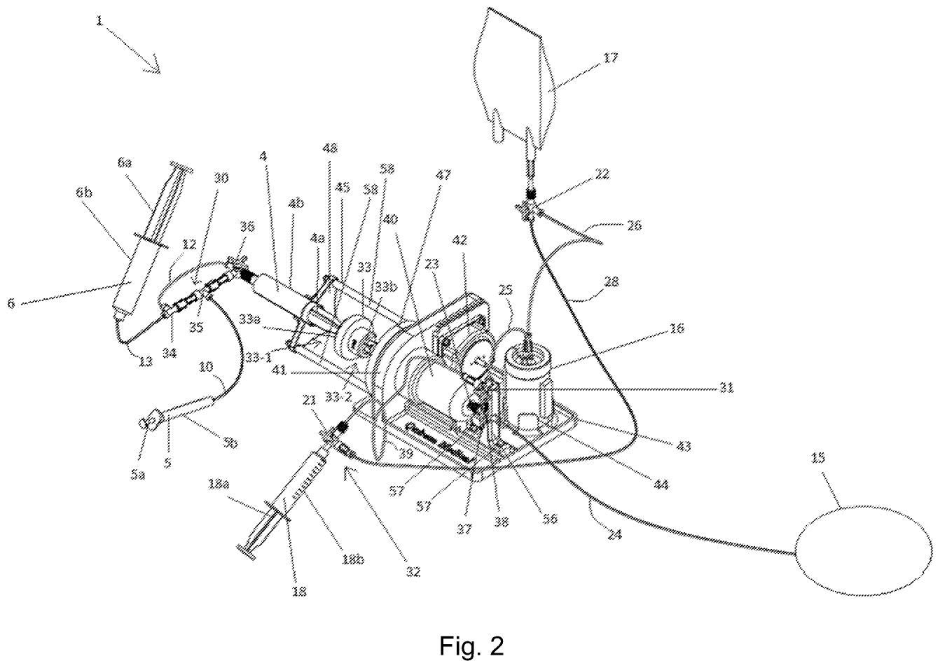

FIG. 2 shows a schematic perspective view of an administration system according to the invention;

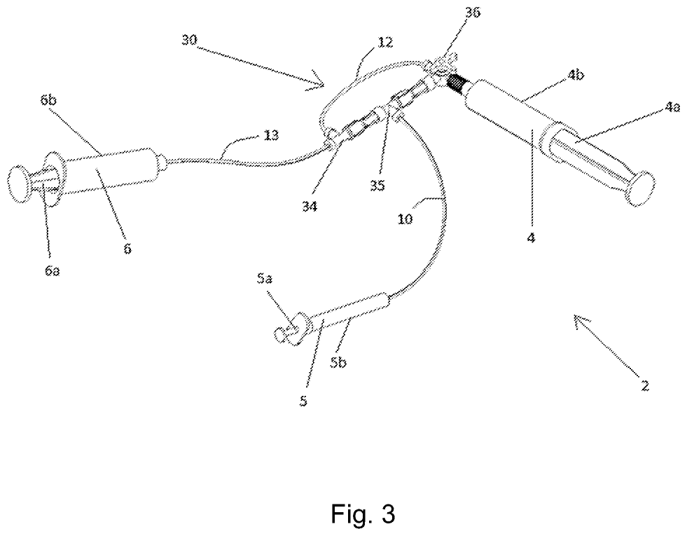

FIG. 3 shows a schematic perspective view of the actuation subsystem;

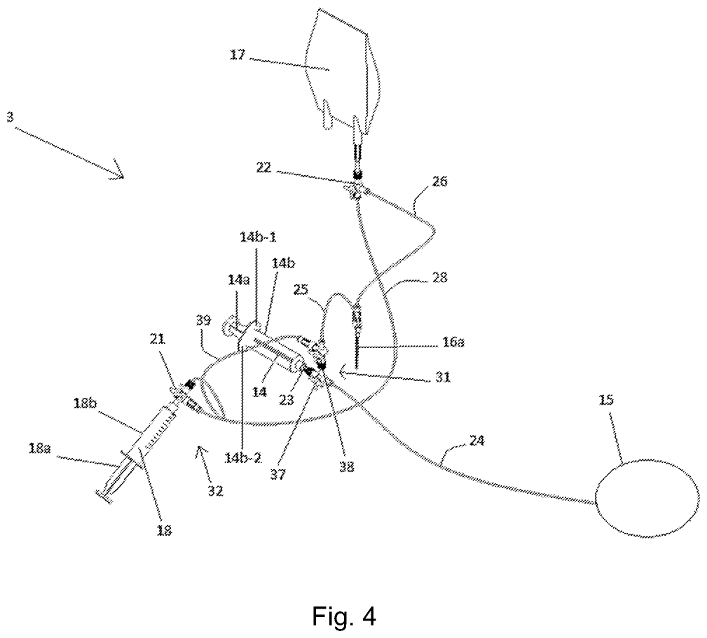

FIG. 4 shows a schematic perspective view of the administration subsystem;

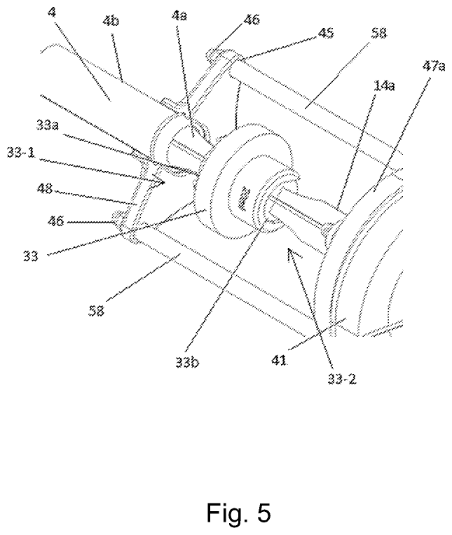

FIG. 5 shows a schematic perspective view of the interface between the actuation subsystem and the administration subsystem;

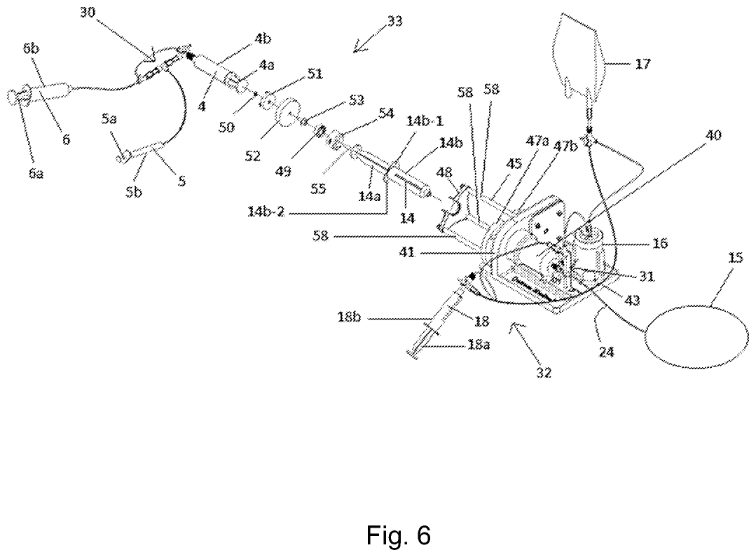

FIG. 6 shows an exploded perspective view of the interface between the actuation subsystem and the administration subsystem;

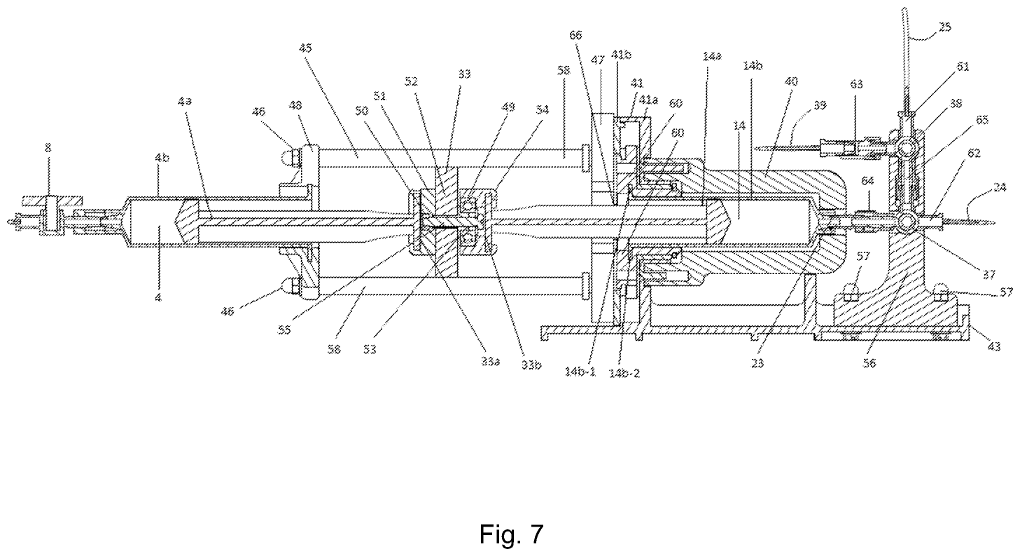

FIG. 7 shows a cross-sectional view of the interface between the actuation subsystem and the administration subsystem in an assembled state;

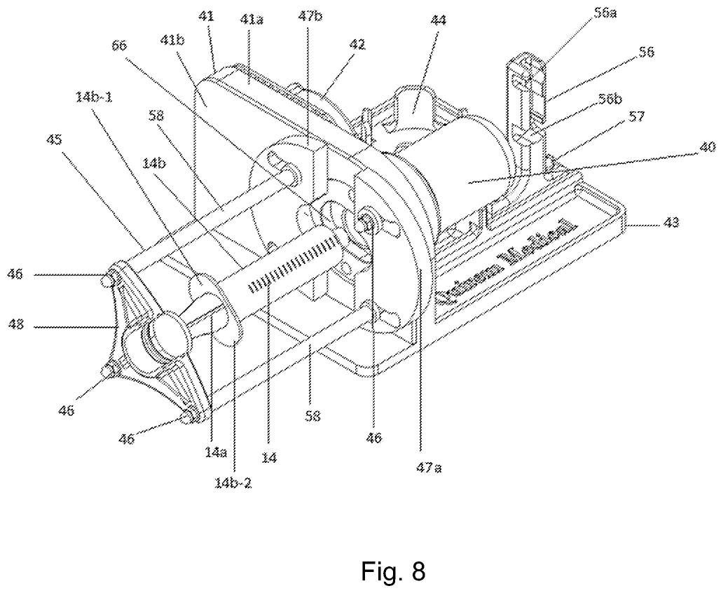

FIG. 8 shows a schematic perspective view of the support frame and the support extension;

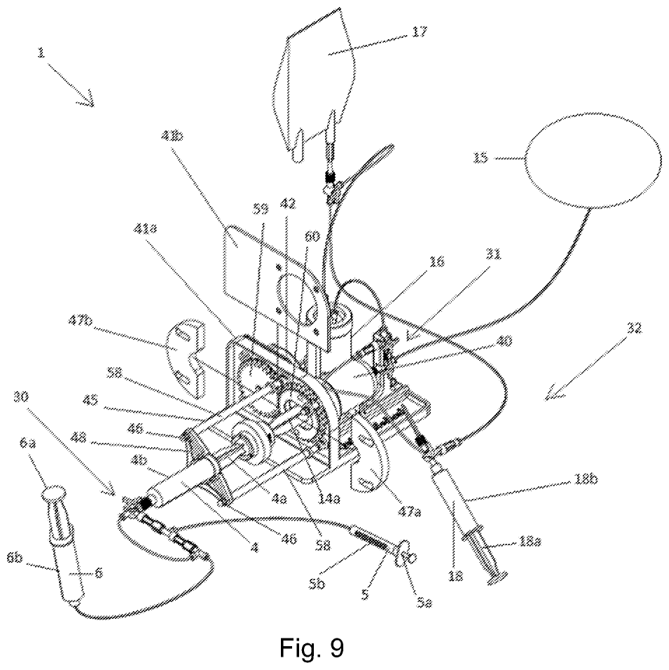

FIG. 9 shows an exploded perspective view of the drive cover and corresponding fastening plate;

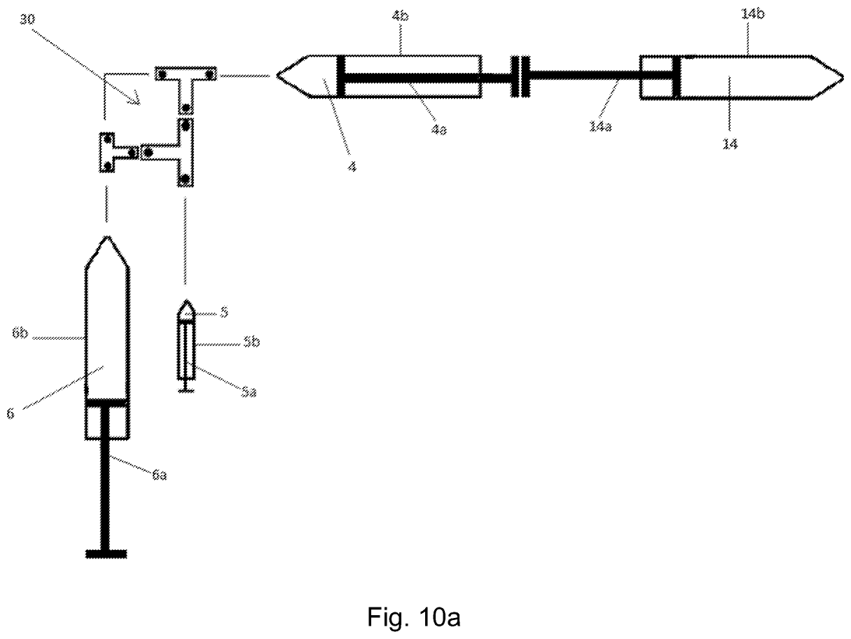

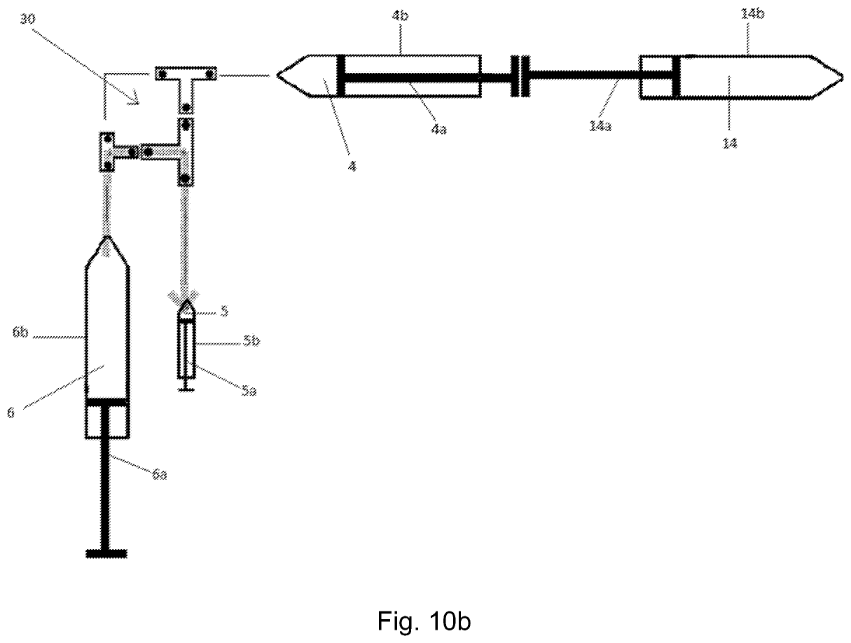

FIG. 10a shows a schematic diagram of the administration system prepared for administration of the particles contained in the administration hydraulic element.

FIG. 10b shows a schematic diagram of the path along which fluid travels in the actuation subsystem during preparation of the administration system for administration.

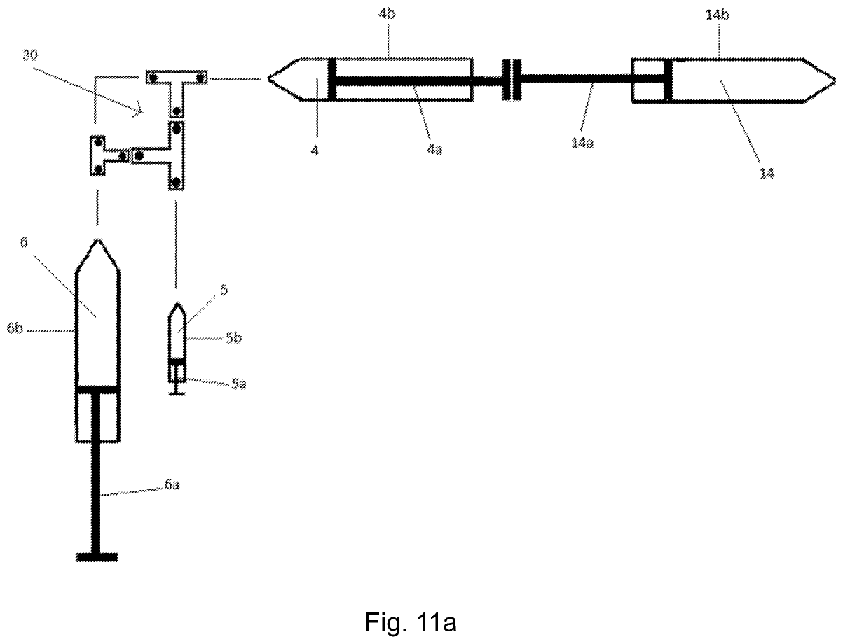

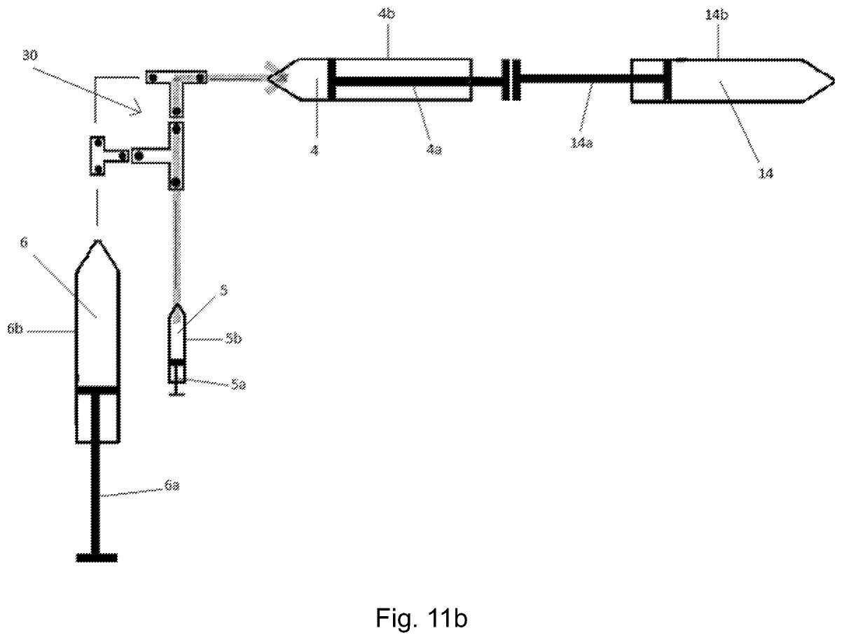

FIG. 11a shows a schematic diagram of the administration system after filling the operating hydraulic element by sucking fluid from the dosage hydraulic element.

FIG. 11b shows a schematic diagram of the path along which fluid travels in the actuation subsystem during actuation of the administration subsystem for administration of the particles.

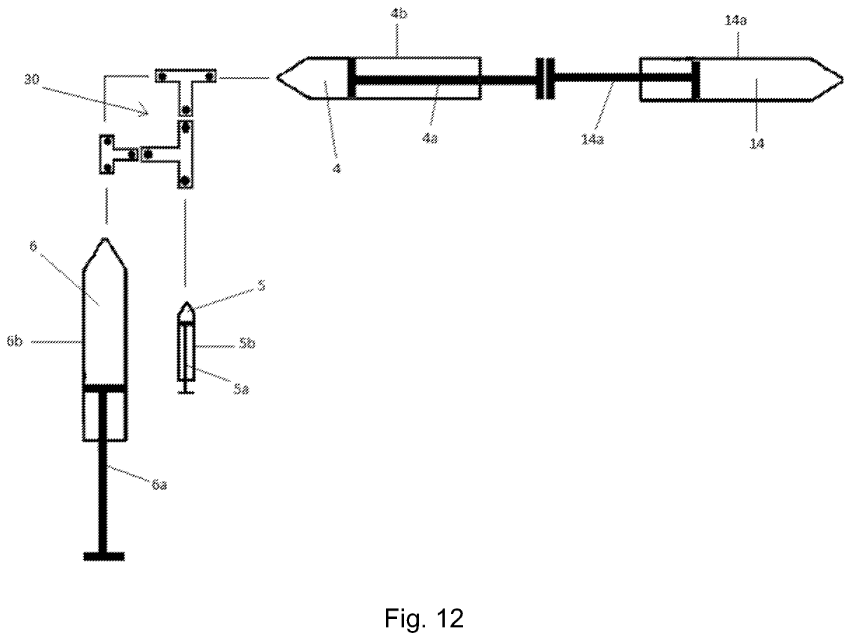

FIG. 12 shows a schematic diagram of the administration system after actuation of the actuation hydraulic element and administration hydraulic element by the operating hydraulic element.

It is to be noted that the figures are given by way of exemplary examples and are not limiting to the disclosure. The drawings may not be to scale. Corresponding elements are designated with corresponding reference signs.

FIG. 1 shows a schematic overview of an administration system 1 according to the invention. The administration system 1 comprises an actuation subsystem 2 and an administration subsystem 3. The actuation subsystem 2 controls the administration subsystem 3 by means of actuation for administering particles originating from a vial 16 to a further system 15.

The actuation subsystem 2 comprises an actuation hydraulic element, e.g. an actuation syringe 4 comprising a plunger 4a and a barrel 4b, to actuate the administration subsystem 3. The actuation subsystem 2 further comprises an operating element, e.g. an operating syringe 5 comprising a plunger 5a and a barrel 5b, for operating the actuation syringe 4. A dosage hydraulic element, e.g. a dosage syringe 6 comprising a plunger 6a and a barrel 6b, may also be part of the actuation subsystem 2. The operating syringe 5 can have a volume of e.g. 1 ml. The actuation syringe 4 and the dosage syringe 6 can have a volume of e.g. 20 ml.

The actuation syringe 4, the operating syringe 5 and the dosage syringe 6 are fluidly connected by an actuation valve unit 30, here formed by connectors 7, 8, 9 and fluid lines 10, 11, 12, 13 such that, in use, actuation of the operating syringe 5 can be transferred to the actuation syringe 4 and feedback concerning administered volume can be provided to an operator via the dosage syringe 6. The connectors 7, 8, 9 and the fluid lines 10, 11, 12, 13 together form an actuation valve unit 30 of the administration system. The connectors 7, 8, 9 each comprise three legs indicated by the reference numeral of the respective connector, followed by one of the letters ‘a’, ‘b’ and ‘c’, wherein when regarding the connector oriented as an upright T-shape, ‘a’ indicates the right leg, ‘b’ indicates the middle leg and ‘c’ indicates the left leg. Each one of connectors 7, 8, 9 can be a three-way valve, T-connector, three-way check valve or other type of three-way hydraulic component.

Other configurations forming an actuation valve unit 30, with more or less fluid lines and connectors, and with types of connectors other than three-way connectors, such as one-way valves, are also envisaged. Another embodiment of the actuation valve unit 30 comprising two check valves 34, 35 and a three-way stopcock 36 is also shown in FIG. 2. In order to enable the operating syringe 5 to actuate actuation syringe 4, a flow path is provided by the actuation valve unit 30 for fluid inside the operating syringe 5 to pass consecutively through fluid line 10, leg 7a of connector 7, leg 7b of connector 7, leg 8b of connector 8, leg 8a of connector 8 and fluid line 11, thus reaching the actuation syringe 4.

A flow path from the operating syringe 5 through fluid line 10, legs 7a and 7b of connector 7, legs 9b and 9c of connector 9 and through flow line 13 to reach dosage syringe 6 is also provided to allow fluid transfer between operating syringe 5 and dosage syringe 6, e.g. for filling the operating syringe 5 before actuating actuation syringe 4. Since providing feedback using dosage syringe 6 is an optional feature of the invention, this pathway can also be left out of the actuation valve unit 30.

A flow path connecting actuation syringe 4 with dosage syringe 6 exists from actuation syringe 4 through fluid line 11, through legs 8a and 8b of connector 8, through legs 7c and 7b of connector 7, through legs 9b and 9c of connector 9, through fluid line 13 into dosage syringe 6. Alternatively, via by-pass fluid line 12, an additional, equivalent yet shorter path is created from actuation syringe 4 through fluid line 11, legs 8a and 8c of connector 8, through fluid line 12, legs 9a and 9c of connector 9, through fluid line 13 to dosage syringe 6. This path can be used to reduce the number of steps for operating the administration system 1.

Administration subsystem 3 comprises an administration hydraulic element, e.g. administration syringe 14 comprising a plunger 14a and a barrel 14b, being rotatably arranged on its output side at a rotatable connector 23 in order to enable rotation of the administration syringe 14 for homogenization of a mixture contained therein. Other types of movement, such as swiveling or translation, with one or more corresponding rotatable connectors 23 or other types of bearing elements to enable this are also possible.

Fluid can flow from the administration syringe 14 to the further system 15 through legs 19a, 19c, 20c, 20a of connectors 19 and 20 and fluid line 24 for administration to the further system 15. A further system 15 could also directly be connected to the administration valve unit, e.g. with connector 20, thus eliminating the need for fluid line 24r.

The administration syringe 14 can be loaded with radioactive particles from a vial 16 by sending a fluid, e.g. a saline solution, contrast agent, fluid containing drug, PBS or any other fluid that might be administered in conjunction with the to be administered particles, contained in reservoir 17 through fluid line 27, legs 22c and 22b of connector 22, through fluid line 26 and into the vial 16 and then further into fluid line 25, legs 19b and 19a of connector 19, through rotatable connector 23 into the administration syringe 14. Passing through rotatable connector 23 can be eliminated if arranged completely at the outside of syringe 14. In FIG. 1, rotatable connector 23 is arranged as an end part of the syringe 14 such that it forms part of the fluid path. It is also envisaged that the solution and particles are already mixed together in the vial, which removes the need for loading from a reservoir 17.

When administering particles, such as radioactive particles, to a patient, administration can be executed in small cycles during which a part of the to be administered dose is administered. The administration subsystem 3 also comprises a flushing system comprising means for flushing the administration subsystem 3 and the further system 15. Accommodating intermittent flushing cycles, during which the solution originating from reservoir 17 is being sent through the administration subsystem without passing through the vial 16 so as not to contain any residual radioactive particles, can be required for safety reasons and a final flushing cycle may be obliged in order to remove residual radioactive particles from the administration system. Flushing can be administered using a flushing hydraulic element, e.g. flushing syringe 18 comprising a plunger 18a and a barrel 18b. The flushing system in the embodiment of FIG. 1 comprises the reservoir 17, fluid lines 27, 28, 29, connectors 21, 22 and flushing syringe 18. A second fluid bag can also be provided, so that the vial 16 and flushing syringe 18 each have their own reservoir to their disposal. In that case, connector 22 can be obviated wherein one bag would be used for fluidizing the radioactive particles and the other would be used as a reservoir for the flushing syringe 18.

Analogously to the actuation valve unit 30 of the actuation subsystem 2, the administration subsystem 3 comprises an administration valve unit 31 that provides the different flow connections and directions between the administration syringe 14, the further system 15 and the vial 16 through fluid lines 24, 25 and connectors 19, 20. Each one of connectors 19, 20 can be a three-way valve, T-connector, three-way check valve or other type of three-way hydraulic component. Other configurations forming an administration valve unit 31 providing the same or similar connections, using more or less fluid lines and connectors, and with types of connectors other than three-way connectors, such as one-way valves, are also possible. Another embodiment of the administration valve unit 31 comprising two three-way stopcocks 37, 38 is also shown in FIG. 2. Stopcock 37 enables a choice between a path to draw flushing fluid from the flushing syringe 18 or a path towards the vial 16 for drawing the particle mixture from the vial 16. Stopcock 38 enables insertion of a fluid or mixture into administration syringe 14 and enables a path from administration syringe 14 towards the further system 15. The flushing fluid drawn from the flushing system may also be envisaged to be able to flow directly through administration valve unit 31, e.g. through stopcocks 37, 38, towards the further system, without passing through the administration syringe 14.

The actuation subsystem 2 and the administration subsystem 3 share an operable connection 33 through which actuation is transferred from the actuation subsystem, originating from an initiation at the operating syringe 5, through the administration subsystem 3 to the further system 15.

FIG. 2 depicts a perspective view of an embodiment of the administration system according to the invention.

As mentioned above, the actuation valve unit 30 and the administration valve unit 31 can be of a variable arrangement. In FIG. 2, the actuation valve unit 30 comprises check valves 34, 35, three-way stopcock 36 and fluid lines 10, 12, 13. The administration valve unit 31 comprises three-way stopcocks 37, 38 and fluid lines 24, 25. Other hydraulic connecting components and configurations thereof are also possible.

In the embodiment of FIG. 2, the flushing system 32 comprises flushing syringe 18, reservoir 17, three-way stopcocks 21, 22 and fluid lines 28, 39.

The administration syringe 14 is held in a receiving container 40 for shielding and/or stabilization purposes. The receiving container 40 can be provided with a shielded wall against radio-active radiation. The receiving container 40 is arranged such as to allow the administration syringe 14 held therein to rotate in order to homogenize the mixture that the administration syringe 14 is containing, in cooperation with the rotatable connector 23. In case no radio-active particles are used, the receiving container can be obviated, and the syringe 14 can be held by other means.

To enable regular rotation of the administration syringe 14, a drive unit 42, powered by a battery pack (not shown) to reduce grid noise influences, is connected to the administration syringe 14. The drive unit 42 can be connected to the administration syringe 14 directly. An indirect connection is also possible, as shown e.g. in FIG. 2, via a drive cover 41, which can be arranged perpendicularly to the longitudinal direction of administration syringe 14 and a rotatable shaft of the drive unit 42 with said directions being arranged substantially parallel, the drive cover 41 thus connecting administration syringe 14 and drive unit 42.

A support frame 43 is provided for holding the administration syringe 14, the vial 16 and the drive unit 42, which together form the center of the administration subsystem 3 and are ideally kept in a stable position fixed in relation to each other to avoid accidents. The support frame 43 can be established as a plate-like element on which parts of the administration subsystem can be provided. The drive cover 41 can be arranged as a support wall, extending upwardly from the support frame. The drive cover 41 can be arranged to cover a drive train connecting the drive unit 42 with the syringe 14 to be driven. As such, a compact arrangement of administration subsystem 3 and drive unit with drive train can be provided. Additionally, the drive cover 41 can function as an additional shielding, e.g. for radio-active radiation, in case radio-active particles are used. The drive cover 41 can thereto be provided from suitable shielding material.

On the support frame 43, a vial holder 44 may further hold the vial 16 to improve stability and/or safety. In FIG. 2, the vial holder 44 is formed comprising vertically extending flanges that may clamp around the vial 16. Other constructions, such as a cylindrically or beam shaped vial holder 44, or other designs, are also envisaged, e.g. a container in which the vial can be received. Extra shielding against radio-activity can also be provided to the vial holder 44. Executing the vial holder 44 in a shape wherein the vial holder 44 extends further away from the support frame 43, perpendicularly to the support frame 43, might be advantageous to provide more shielding of the vial 16.

The support frame 43 also comprises a connector holder 56 for holding connectors of the administration valve unit 31, in this embodiment for holding stopcocks 37, 38 vertically on top of each other.

The operable connection 33 between the actuation syringe 4 and the administration syringe 14 is arranged as a coupling element 33, comprising a first engagement surface 33a at a first side 33-1 of the coupling element 33 and a second engagement surface 33b at a second side 33-2, opposite to the first side 33-1, of the coupling element 33. The first side 33-1 corresponds with an actuation subsystem side of the administration system and the second side 33-2 corresponds with an administration subsystem side of the administration system. Forming the interface between actuation subsystem 2 and administration subsystem 3, the first engagement surface 33a is intended for connection with the actuation syringe 4, while the second engagement surface 33b is intended for connection with the administration syringe 14. A bearing at its first engagement surface 33a is provided to the coupling element 33, the bearing preventing rotational movement of actuation syringe 4. In addition, the coupling element 33 comprises a bearing at its second engagement surface 33b that allows for rotational movement of administration syringe 14. Coupling element 33 can be arranged as a disc-shaped component comprising a ceramic bearing at its second engagement surface 33b for an increased wear-resistance.

It shall be appreciated that various alternatives to the above-described configuration for the operable connection are possible, for example without the described coupling element. As indicated elsewhere herein, in a more general sense, the administration and actuation hydraulic elements are advantageously operably connected, e.g. mechanically connected, such as (partially) directly pressed together or connected by interconnection means, electronically connected or connected in another way.

Keeping the actuation syringe 4 and especially the interface between the actuation syringe 4 and the administration syringe 14 in place, a support extension 45 can be installed adjacent to the support frame 43. By providing a support extension 45 as a number of parallel rods 58, e.g. three parallel rods 58, approximately along the circumference of a circle, transverse to the longitudinal direction of the administration syringe 14 and with its center along that longitudinal direction of the administration syringe 14, an operator can visually monitor the correct engagement at the interface of actuation subsystem 2 and administration subsystem 3 during testing and/or operation of the administration system 1, while keeping the interface between the actuation subsystem 2 and the administration subsystem 3 stabilized.

The support extension 45 further comprises a fastening plate 47 for connecting the support extension 45 with the support frame 43 through drive cover 41 and a fastening plate 48 comprising an actuation syringe receiving opening for holding the actuation syringe barrel 4b. Fastening plates 47, 48 keep the rods 58 parallel to each other and substantially parallel to the support frame 43. Support extension 45 and support frame 43 could also be arranged making an angle.

At its proximal end, the administration syringe 14 can further be shielded by providing drive cover 41 and/or fastening plate 47 with shielding material that blocks radio-activity for increased containment of the radio-activity originating from the mixture inside the administration syringe 14. Of course, if no radio-active particles are envisaged to use, shielding material to shield from radio-activity can be obviated, as well as that the receiving container can be obviated as well. When no shielding for radio-activity is needed, the receiving container may be replaced by for example a simple holding element.

FIGS. 3 and 4 show embodiments of the actuation subsystem 2 and the administration subsystem 3 respectively.

The actuation subsystem of FIG. 3 shows the principal components for actuating the plunger 4a of actuation syringe 4, as mentioned above. The operating syringe 5 can e.g. be a 1 ml syringe which adds to a high-precision administration system, seeing that actuation syringe 4 can e.g. be a 20 ml syringe. In order to be able to read from dosage syringe 6 the volume that has been moving actuation syringe plunger 4a thus far during administration, dosage syringe barrel 6b contains a volume of e.g. at least 20 ml. This enables a corresponding volume of actuation syringe 4 to be withdrawn from the dosage syringe 6 during administration steps. Further details on administration steps are provided in FIGS. 10a, 10b, 11a, 11b and 12.

FIG. 3 shows an embodiment for the actuation valve unit 30 as in FIG. 2. However, as explained above, the actuation valve unit 30 can have various configurations, e.g. as illustrated in FIG. 1. Flexible fluid lines 10, 12, 13 can preferably be filled with a fluid of low compressibility, to enable a translation of plunger 5a of the operating syringe 5 to correspond with a same, or almost identical displacement of plunger 4a of the actuation syringe 4 along longitudinal directions of said syringes, such as water, but may also be filled with other fluids or mixtures. It is also envisaged in the invention that a more compressible fluid may be used, wherein a volume displaced by plunger 5a from barrel 5b of operating syringe 5, could correspond with a displacement of a smaller volume in barrel 4b of actuation syringe 4. Advantageously, a fluid might be chosen such that a specific ratio of displaced volumes is obtained that can increase administration precision or provide a precision comparable to an implementation using a non-compressible fluid.

Fluid lines 10, 12, 13 may be flexible fluid lines, or non-flexible fluid lines. In an embodiment, flexible fluid lines are used for a less rigid implementation, which introduces flexibility in the actuation subsystem as such. Non-flexible flow lines are envisaged e.g. for industrial applications, wherein circumstances call for uniformity along the system.

FIG. 4 depicts the administration subsystem 3. The plunger 14b of administration syringe 14 is actuated from the actuation subsystem 2, after a mixture of a fluid originating from reservoir 17 with radio-active particles from vial 16 have been transported into administration syringe 14 through needles 16a, flexible flow line 25, stopcocks 37, 38 and rotatable connector 23, and after the administration syringe 14 has been brought into a e.g. rotational movement to homogenize the particles. Needles 16a on the inside of vial 16 are shown, for accessing the radioactive particles to introduce them into the fluid lines of the administration subsystem 3. The vial 16 itself is not shown in order to expose the needles 16a in this view.

Actuation syringe 14 may also comprise a first protruding portion 14b-1 and a second protruding portion 14b-2 at the end of its barrel 14b for engaging with a rotational component for enabling the administration syringe 14 to rotate for homogenizing the mixture contained in the administration syringe 14. Administration syringe 14 may also comprise other, protruding, intruding or other components for allowing engagement with a rotating element.

As mentioned above, the administration valve unit 31 can comprise numerous configurations and fluid and particles can be drawn into administration syringe 14 through other paths formed by different components. FIG. 3 repeats the configuration shown in FIG. 2, however, other configurations, as e.g. in FIG. 1 or otherwise, are also possible.

FIG. 5 depicts a schematic perspective view of the interface between the actuation subsystem 2 and the administration subsystem 3, comprising an enlarged view of the coupling element 33. Here, it can be seen that the coupling element 33 is a separate, virtually floating component that is engaged at the first side 33-1 with the plunger 4a of the actuation hydraulic element 4 through the first engagement surface 33a and at the second side 33-2 with the plunger 14a of the administration hydraulic element 14 through the second engagement surface 33b. The coupling element 33 has no further connection, so when removing either one of the plungers 4a, 14a, the coupling element 33 may come loose. The coupling element 33 components are shown in FIGS. 6 and 7.

The rods 58 are fastened to fastening plates 47, 48 by fastening means 46, which can e.g. comprise bolts. The fastening means 46 need not be identical.

Fastening plate 47 may comprise two symmetrical, ring-section-like shaped fastening plates, first section fastening plate 47a and second section fastening plate 47b. This is further illustrated in FIGS. 6, 8 and 9.

FIGS. 6 and 7 show views of the coupling element 33 between the actuation syringe 4 and the administration syringe 14. On the side of the administration subsystem 3, bearing 49 allows for rotation of the administration syringe 14 around its longitudinal center axis. Bearing 49 is held by bearing holding element 53, which is placed against disc element 52 and fixated therein by screw 55. Disc element 54 is placed around the bearing 49 and administration syringe 14 to hold the administration syringe 14 by e.g. press-fitting. On the side of the actuation subsystem 2, disc element 51 is placed against disc element 52 and screw 55 reaches through disc elements 51, 52 to press them together. Screw end holder 50 is arranged at the center of disc element 51 to fasten screw 55 at its distal end. The disc element 52 is formed such that it can hold the plunger of actuation syringe 4 by a press-fitting connection.

A cross-section of the interface between the actuation subsystem 2 and the administration subsystem 3 is shown in FIG. 7, showing the coupling element 33 in an assembled state. FIG. 7 is a cross-section along a central axis of the syringes 4, 14 of the administration system 1. FIG. 7 also illustrates how the support extension 45 could be fastened to the frame 43. The ends of the rods 58 of the support extension 45 are bolted into fastening plates 47, 48 to obtain a stable structure. Fastening plate 47 is provided with a receiving opening 66 for receiving the administration syringe 14, allowing translation of the syringe plunger 14a through the opening when the plunger 14a is moved by the actuation plunger 4a. The fastening plate 47 is shown in more detail in FIGS. 8 and 9. In this example, the support extension 45 is fastened to the drive cover 41 mounted to the support frame 43.

Drive cover 41 comprises a drive cover main part 41a and a drive cover closing part 41b, surrounding drive element 60. Drive element 60 engages with the protruding portions 14b-1, 14b-2 of the administration syringe 14, such that barrel 14b of administration syringe 14 is rotated along with drive element 60. Drive element 60 can e.g. have a ring-like shape arranged around syringe barrel 14b.

Drive element 60 can be drivingly coupled to drive unit 42, such that the drive element 60 is driven by the drive unit 16, simplifying installation and maintenance of the administration system 1.

Fluid lines 24, 39 are only partially shown in FIG. 7 for conciseness. As mentioned earlier, administration valve unit 31 may have various embodiments. Stopcocks 37, 38 are here depicted as comprising stopcock ends 61, 62, 63, 64 and stopcock-stopcock interface 65 for stable and easily controllable paths. Other implementations are also possible.

Connector holder 56 is depicted here as having a T-like cross-section shape. Other shapes, such as an I-shape or an L-shape, are also envisaged.

FIG. 8 shows a schematic perspective view of the support frame 43 and the support extension 45. Support frame 43 comprises a connector holder 56, connected to the support frame 43 by fastening means, e.g. bolts 57, to hold the connector elements 37, 38. Here, it is shown how a syringe 14 is inserted in the receiving container 40. An opening 66 in the drive cover 41 is opening by the frame portion 47b. The syringe 14 can be inserted through the opening 66 into the receiving container 40. Plunger portions 14b-1, 14b-2 can then engage with a drive element 60, shown in FIG. 9, that is drivingly connected to the drive unit 2. Thus, the syringe 14 is held in the receiving container 40. The frame portion 47b can then be closed, thus providing a shielding to any radio-active material that may be provided in the administration subsystem 3.

FIG. 9 is an exploded perspective view of the drive cover 41. The drive cover 41 comprises main part 41a and cover part 41b. The support wall 41 can be an integral part of the support frame 43, and may be arranged in an upright manner with respect to the support frame 43.

The drive unit 42 provides means for rotating the administration syringe 14. This can be done by directly engaging a part of e.g. a rotating shaft at a circumference of the administration syringe 14. In FIG. 9, another embodiment is shown, wherein drive unit 42 engages actuation syringe 14 through engagement with a drive train 59 further to a drive element 60, which engages protruding portions 14b-1, 14b-2 of administration syringe 14. Drive train 59 and drive element 60 are shown here as gears, but can also be implemented using other transmission means, such as e.g. a belt transmission.

FIGS. 10a, 10b, 11a, 11b and 12 illustrate the steps to be taken for administration of a volume inside the operating element 5. Here, actuation valve unit 30 is again depicted as in FIG. 1, but as mentioned above, actuation valve unit 30 can take any other form or configuration that enables the abovementioned hydraulic connections between the actuation syringe 4, the operating syringe 5 and the dosage syringe 6.

Initially, dosage syringe 6 is in a filled state and operating syringe 5 and actuation syringe 4 are in an emptied state, see FIG. 10a. To initiate the system, the syringe 14 is brought into rotation by the drive unit 2. After a predetermined amount of time, the suspension in the syringe 14 is in rotation as well, and can be said to be homogenous. To start administration, a volume of fluid is sucked from dosage syringe 6 into operating syringe 5 by moving plunger 5a to reach the state shown in FIG. 11a, by moving liquid along a path as shown in FIG. 10b.

Subsequently, the content of operating syringe 5 can be directed along a path towards actuation syringe 4, see FIG. 11b, by manually operating the operating syringe 5 by again moving plunger 5a, this time pushing fluid inside operating syringe 5 out of operating syringe 5, until the operating syringe 5 is emptied.

Finally, the state as shown in FIG. 12 is reached, upon which a new cycle of administering a volume inside operating syringe 5, e.g. 1 ml, can be started to further move the plunger 4a of actuation syringe 4, possibly until actuation syringe 4 is fully filled, e.g. with 20 ml of water. Now, it is known that a volume of suspension from the syringe 14 equal to the volume of the operating syringe 5 is inputted into the further system. Also, an operator can accurately move the plunger 5a of the operating syringe 5, e.g. in multiple steps. At each step, the operator knows that a volume equal to the volume of fluid pushed out of the operating syringe 5 with the plunger 5a is via, the actuation syringe 4 and the administration syringe 14, inputted into the further system 15. So, the operator accurately knows how much of the suspension is inputted in the further system. Also, because the suspension of the particles in the syringe 14 is homogenous due to the rotation thereof, the operator accurately knows the volume of particles inserted into the further system 15. The operator can, in one or multiple steps, empty the operating syringe 5. After emptying the operating syringe 5, it can be filled again by opening the fluid connection between the dosage syringe 6 and the operating syringe 5 as shown in FIG. 10b. As such, the dosage syringe 6 can give a read-out of the volume already inserted in the further system 15. Once the suspension is in the further system 15, e.g. a catheter connected to a patient the suspension can be flushed into the patient by means of flushing fluid that via the valve unit 31 can be inserted into the further system 15. As explained above, the syringe 14 is first filled with the particles from the vial 16 by using fluid from the container 17.

As shown in the embodiments of FIGS. 10a to 12, plungers 4a and 14a can also be simply pressed together, without coupling element 33, enabling plunger 14a not to rotate, contrary to other embodiments of the invention, wherein plunger 14a rotates with syringe 14.

It will be appreciated that many variants of the various components of the system are possible. Some of those variants are described above.

For the purpose of clarity and a concise description, features are described herein as part of the same or separate embodiments, however, it will be appreciated that the scope of the claims and disclosure may include embodiments having combinations of all or some of the features described. It may be understood that the embodiments shown have the same or similar components, apart from where they are described as being different.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage. Many variants will be apparent to the person skilled in the art and are understood to be comprised within the scope of the invention defined in the following claims.

The present disclosure comprises the following numbered embodiments E1-E50.
E1. A system for administration of particles, the system comprising:
   an administration subsystem comprising at least one administration hydraulic element for containing a mixture with the particles, wherein the at least one administration hydraulic element is movably mounted for, when moving, homogenizing the mixture, wherein said at least one administration hydraulic element is arranged for administering of the mixture to a further system;
   an actuation subsystem comprising at least one actuation hydraulic element; wherein the at least one actuation hydraulic element is operably connected to the at least one administration hydraulic element for actuating said administration hydraulic element to administer mixture to the further system; wherein the at least one actuation hydraulic element is further fluidly connected with an operating element to operate the at least one actuation hydraulic element.
E2. The system according to E1, wherein the actuation subsystem further comprises a dosage hydraulic element fluidly connected to the at least one actuation hydraulic element.
E3. The system according to E1 or E2, wherein the actuation subsystem further comprises an actuation valve unit, the actuation valve unit fluidly connecting the operating element and the at least one actuation hydraulic element and/or the dosage hydraulic element.
E4. The system according to E3, wherein the actuation valve unit comprises at least one three way valve.
E5. The system according to E3 or E4, wherein the actuation valve unit comprises a bypass line between the at least one actuation hydraulic element and the dosage hydraulic element to allow a direct fluid connection between the at least one actuation hydraulic element and the dosage hydraulic element.
E6. The system according to any of E1-E5, wherein the operating element is a hydraulic element.
E7. The system according to E6, wherein the operating element has a volume smaller than a volume of the at least one actuation hydraulic element, preferably has a volume that is about 10 times smaller than the volume of the actuation hydraulic element, more preferably has a volume that is about 20 times smaller than the volume of the actuation hydraulic element.
E8. The system according to any of E2-E7, wherein the dosage hydraulic element has a volume that is the same or larger than a volume of the at least one actuation hydraulic element.
E9. The system according to any of E1-E8, wherein the operating element and/or the dosage hydraulic element are manually operable.
E10. The system according to any of E1-E9, wherein the actuation subsystem comprises flexible flow lines for connection between at least the at least one actuation hydraulic element, the operating element and/or the dosage hydraulic element.
E11. The system according to any of E1-E10, wherein the operating element is fluidly connected to the at least one actuation hydraulic element via a flexible flow line, wherein the flexible flow line is at least 0.2 m, preferably more than 0.5 m.
E12. The system according to any of E1-E11, wherein the at least one actuation hydraulic element and/or the dosage element are syringes.
E13. The system according to any of E1-E12, wherein the actuation subsystem is filled with water and/or is re-usable.
E14. The system according to any of E1-E13, wherein the administration subsystem comprises a receiving unit for receiving the at least one administration hydraulic element, wherein the receiving unit is arranged for allowing movement of the at least one administration hydraulic element.
E15. The system according to E14, wherein the receiving unit comprises a receiving container for receiving the administration hydraulic element therein, preferably, wherein a wall of the container is provided as a shield against radio-active radiation.
E16. The system according to E15, wherein in a receiving opening of the receiving container a drive element is provided that is arranged for engaging with the administration hydraulic element.
E17. The system according to any of E14-E16, wherein the at least one administration hydraulic element is rotatably arranged.
E18. The system according to any of E14-E17, wherein the administration subsystem comprises a drive unit for driving the movement of the at least one administration hydraulic element.
E19. The system according to E18 and E16, wherein the drive unit is drivingly coupled to the drive element to drive the drive element.
E20. The system according to any of E14-E19, wherein the administration subsystem comprises a support frame, preferably comprising the receiving unit and the drive unit.
E21. The system according to any of E14-E20, wherein the administration subsystem further comprises at least one holder for holding a vial initially filled with particles, wherein the vial is fluidly couplable with the administration hydraulic element for loading the particles to the administration hydraulic element, preferably wherein the at least one holder is arranged on the support frame.
E22. The system according to any of E1-E21, wherein the administration hydraulic element and/or the vial for containing the mixture with the particles and/or the particles are optimized for a particle diameter, a particle density and/or a particle size, wherein the particles are microspheres, preferably radioactive microspheres, preferably QuiremSpheres, SIR-Speres, TheraSphere or CT imageable microspheres, preferably microspheres labeled with chemotherapeutic agents, such as TACE microspheres, wherein the particles are nanoparticles of 20-1000 nanometer or 1-1000 micrometer, more preferably 1-400 micrometer, more preferably 10-60 or 60-200 micron, or 80-150 micron for embolization of vessels.
E23. The system according to any of E14-E22, wherein the administration subsystem comprises an administration valve unit for providing a fluid connection between the at least one administration hydraulic element, the vial and the further system.
E24. The system according to any of E14-E23, wherein the administration subsystem further comprises a flushing system that is fluidly couplable to the at least one administration hydraulic element, preferably wherein the flushing system is connectable to the administration valve unit.
E25. The system according to E23, wherein the flushing system comprises a flushing hydraulic element and a container containing fluid, wherein the container and the flushing hydraulic element are fluidly connectable to each other, preferably wherein the flushing hydraulic element is connectable to the administration valve unit, preferably wherein the fluid is one of a saline, a contrast agent, a drug containing fluid, a phosphate buffered saline.
E26. The system according to any of E23-E25, wherein the administration valve unit comprises at least one three way valve.
E27. The system according to any of E1-E26, further comprising an operable connection between the at least one actuation hydraulic element and the at least one administration hydraulic element, wherein the operable connection comprises a coupling element.
E28. The system according to E27, wherein the coupling element comprises a first engagement surface for engaging with the actuation hydraulic element, preferably with a piston of the actuation hydraulic element, and a second engagement surface for engaging with the administration hydraulic element, preferably with a piston of the administration hydraulic element, wherein the second engagement surface movable arranged with respect to the first engagement surface.
E29. The system according to E28, wherein the coupling element is a disc shaped element with the first engagement surface at one side and the second engagement surface at the other side of the disc shaped element, preferably wherein the second engagement surface is rotatably arranged with respect to the first engagement surface to allow rotatable movement of the associated administration hydraulic element.
E30. The system according to any of E27-E29, wherein the coupling element is arranged to provide shielding of the administration subsystem, preferably of radio-active radiation of the administration subsystem.
E31. The system according to any of E27-E30, wherein the coupling element is configured as a disc-shaped element comprising a ceramic bearing for allowing rotational movement of the second engagement surface.
E32. The system according to any of E20-E31, wherein the support frame further comprises a support extension, wherein the support extension is arranged to support the at least one actuation hydraulic element for engagement with the at least one administration hydraulic element.
E33. The system according to any of E14-E32, wherein the administration subsystem is MRI-compatible, in particular wherein a drive unit of the administration subsystem is MRI-compatible.
E34. The system according to any of E1-E33, wherein the administration subsystem comprises disposable components.
E35. The system according to any of E1-E34, wherein the administration subsystem comprises a battery pack for powering the drive unit.
E36. The system according to any of E1-E35, wherein the further system is a clinical system, an intravenous system, an intratumoral system, and intra-arterial system or an industrial system for filling multiple vials with the mixture containing particles.
E37. Method for filling multiple vials with a mixture containing particles, the method comprising
   providing a system according to any of E1-E36;
   connecting at least one vial to be filled to the system;
   preparing the at least one administration hydraulic element with the mixture containing the particles;
   moving the said administration hydraulic element until the mixture is homogeneous;
   operating the actuation subsystem to actuate the administration hydraulic element such that a predefined volume of mixture is administered to the further system;
   optionally flushing the mixture through the further system with flushing fluid.
E38. Method for outputting a predetermined volume of particles from a hydraulic element containing a mixture with the particles, the method comprising:
   providing a system according to any of E1-E36;
   preparing the at least one administration hydraulic element with the mixture containing the particles;
   moving the said administration hydraulic element until the mixture is homogeneous;
   operating the actuation subsystem to actuate the administration hydraulic element such that a predefined volume of mixture is outputted of the administration hydraulic element.
E39. A mixture containing particles for use in a method of treating a tumor in an individual, wherein the mixture is administered by a system according to any of E1-E36.
E40. The mixture containing particles for use according to E39, wherein the method comprises intravenous administration, intratumoral administration or intra-arterial administration of the mixture.
E41. The mixture containing particles for use according to E39 or E40, wherein the cancer is a gastro-intestinal cancer, preferably a liver cancer or pancreas tumors, or head and/or neck tumors.
E42. The mixture containing particles for use according to any of E39-E41, wherein the particles are radio-active particles, preferably QuiremSpheres, SIR-Speres, TheraSphere or CT imageable microspheres.
E43. The mixture containing particles for use according to any of E39-E42, wherein the particles are nanoparticles of 20-1000 nanometer or the particles are microspheres of 1-1000 micrometer, preferably 1-400 micrometer, more preferably 10-60 micrometer, more preferably 60-200 micrometer, most preferably 80-150 micrometer for the embolization of vessels.
E44. The mixture containing particles for use according to any of E39-E43, further comprising administering to the individual a chemotherapeutic agent, preferably said chemotherapeutic agent is coupled to said particles.
E45. Method of treating an individual with cancer with a mixture containing particles, the method comprising
   providing a system according to any of E1-E36;
   preparing the at least one administration hydraulic element with the mixture containing the particles;
   moving the said administration hydraulic element until the mixture is homogeneous;
   operating the actuation subsystem to actuate the administration hydraulic element such that a predefined volume of mixture is administered to the individual; and
   flushing the mixture through the further system with flushing fluid; thus administrating the mixture to the individual.
E46. The method according to E45, wherein the method comprises intravenous administration, intratumoral administration or intra-arterial administration of the mixture.
E47. The method according to E45 or E46, wherein the cancer is a gastro-intestinal cancer, preferably a liver cancer.
E48. The method according to any of E45-E47, wherein the particles are radio-active particles, preferably QuiremSpheres, SIR-Speres, TheraSphere or CT imageable microspheres.
E49. The method according to any of E45-E48, wherein the particles are nanoparticles of 20-1000 nanometer or the particles are of 1-1000 micrometer, preferably 1-400 micrometer, more preferably 10-60 micrometer, more preferably 60-200 micrometer, most preferably 80-150 micrometer.
E50. The method to any of E45-E49, further comprising administering to the individual a chemotherapeutic agent, preferably said chemotherapeutic agent is coupled to the particles.

The invention claimed is:

1. A system for administration of particles, the system comprising:
an administration subsystem comprising at least one administration hydraulic element for containing a mixture with the particles, wherein the at least one administration hydraulic element is movably mounted in a movable arrangement for, when moving, homogenizing the mixture, wherein said at least one administration hydraulic element is arranged for administering of the mixture to a further system;
an actuation subsystem comprising at least one actuation hydraulic element; wherein the at least one actuation hydraulic element is operably connected, providing an operable connection, to the at least one administration hydraulic element for actuating said administration hydraulic element to administer mixture to the further system; wherein the at least one actuation hydraulic element is further fluidly connected, providing a fluid connection, with an operating element to operate the at least one actuation hydraulic element,
wherein the actuation subsystem is, hydraulically, decoupled from the administration subsystem.

2. The system according to claim 1, wherein the actuation subsystem further comprises a dosage hydraulic element fluidly connected to the at least one actuation hydraulic element.

3. The system according to claim 2, wherein the actuation subsystem further comprises an actuation valve unit, the actuation valve unit fluidly connecting the operating element and the at least one actuation hydraulic element and/or the dosage hydraulic element.

4. The system according to claim 3, wherein the actuation valve unit comprises at least one three way valve.

5. The system according to claim 3, wherein the actuation valve unit comprises a bypass line between the at least one actuation hydraulic element and the dosage hydraulic element to allow a direct fluid connection between the at least one actuation hydraulic element and the dosage hydraulic element.

6. The system according to claim 1, wherein the operating element is a hydraulic element.

7. The system according to claim 1, wherein the operating element has a volume smaller than a volume of the at least one actuation hydraulic element.

8. The system according to claim 2, wherein the dosage hydraulic element has a volume that is the same or larger than a volume of the at least one actuation hydraulic element.

9. The system according to claim 2, wherein the operating element and/or the dosage hydraulic element are manually operable.

10. The system according to claim 2, wherein the at least one actuation hydraulic element and/or the dosage hydraulic element are syringes.

11. The system according to claim 1, wherein the actuation subsystem is filled with liquid and/or is re-usable.

12. The system according to claim 1, wherein the administration subsystem comprises a receiving unit for receiving the at least one administration hydraulic element, wherein the receiving unit is arranged for allowing movement of the at least one administration hydraulic element.

13. The system according to claim 12, wherein the receiving unit comprises a receiving container for receiving the administration hydraulic element therein and/or wherein in a receiving opening of the receiving container a drive element is provided that is arranged for engaging with the administration hydraulic element.

14. (canceled)

15. The system according to claim 1, wherein the at least one administration hydraulic element is rotatably arranged.

16. The system according to claim 1, wherein the administration subsystem comprises a drive unit for driving movement of the at least one administration hydraulic element.

17. The system according to claim 16, wherein the administration subsystem comprises a receiving unit for receiving the at least one administration hydraulic element, wherein the receiving unit comprises a receiving container for receiving the administration hydraulic element therein, wherein in a receiving opening of the receiving container a drive element is provided that is arranged for engaging with the administration hydraulic element, and wherein the drive unit is drivingly coupled to the drive element that is arranged for engaging with the administration hydraulic element.

18. (canceled)

19. The system according to claim 1, wherein the administration subsystem further comprises at least one holder for holding a vial initially filled with particles, wherein the vial is fluidly couplable with the administration hydraulic element for loading the particles to the administration hydraulic element.

20. (canceled)

21. The system according to claim 1, wherein the administration subsystem comprises an administration valve unit for providing a fluid connection between the at least one administration hydraulic element, a vial and a further system.

22. The system according to claim 1, wherein the administration subsystem further comprises a flushing system that is fluidly couplable to the at least one administration hydraulic element and/or wherein the flushing system comprises a flushing hydraulic element and a container containing fluid, wherein the container and the flushing hydraulic element are fluidly connectable to each other.

23. (canceled)

24. (canceled)

25. The system according to claim 1, wherein the operable connection between the at least one actuation hydraulic element and the at least one administration hydraulic element comprises a coupling element.

26. The system according to claim 25, wherein the coupling element comprises a first engagement surface for engaging with the actuation hydraulic element, and a second engagement surface for engaging with the administration hydraulic element, wherein the second engagement surface movable arranged with respect to the first engagement surface.

27. The system according to claim 25, wherein the coupling element is arranged to provide shielding of the administration subsystem.

28. (canceled)

29. The system according to claim 1, wherein the administration subsystem is MRI-compatible.

30. (canceled)

31. (canceled)

32. (canceled)
